SEQUENCE LISTING (2 sequences)

SEQ ID NO: 1 - protein, 5 aa, Artificial sequence
  Chemically synthesized
ALKHR

SEQ ID NO: 2 - protein, 20 aa, Artificial sequence
  Chemically synthesized
FCTGIRVAHLALKHRQGKNH

(19) United States
(12) Patent Application Publication
(10) Pub. No.: US 2017/0174734 A1
(43) Pub. Date: Jun. 22, 2017

(54) DR6 Receptor Mediates the Leukemia Differentiation Activity of Angiocidin: A Potent Anti-Tumor Peptide

(71) Applicant: Temple University-Of The Commonwealth System of Higher Education, Philadelphia, PA (US)

(72) Inventor: George P. Tuszynski, Pittsgrove, NJ (US)

(21) Appl. No.: 15/125,384

(22) PCT Filed: Mar. 12, 2015
(86) PCT No.: PCT/US15/20134

§ 371 Date: Sep. 12, 2016

Related U.S. Application Data
(60) Provisional application No. 61/951,625, filed on Mar. 12, 2014.

(51) Int. Cl. C07K 14/47; G01N 33/574; C07K 16/18

(52) U.S. Cl. CPC C07K 14/4747; C07K 16/18; G01N 33/57426; G01N 33/5743; G01N 33/57415; G01N 33/57407; A61K 38/00

(57) ABSTRACT

The present invention provides compositions and methods of treating cancer by inducing the cellular differentiation activity of angiocidin.

### CROSS-REFERENCE TO RELATED APPLICATIONS

This application claims priority to U.S. Provisional Application Ser. No. 61/951,625, filed Mar. 12, 2014, the content of which is incorporated by reference herein in its entirety.

### BACKGROUND OF THE INVENTION

Angiocidin is a protein, originally isolated from lung carcinoma that is overexpressed in many tumor systems (Zhou et al., J. Cell. Biochem. 92: 125-146, 2004; Poon, et al., Clin. Cancer Res. 12: 4150-4157, 2004). Angiocidin is a receptor for thrombospondin-1 and is a potent inhibitor of angiogenesis and tumor cell proliferation (U.S. 2003/0180295; Zhou et al., J. Cell. Biochem. 92: 125-146, 2004). These functions of angiocidin are mediated by α2β1 integrin (Sabherwal, et al., Exp. Cell Res. 312: 443-453, 2006). In addition, angiocidin has important immunomodulatory effects on monocytes that can affect the course of disease.

Acute myeloid leukemia (AML) is cancer of the myeloid line of blood cells. AML is marked by the production of irregular white blood cells. Production of these abnormal white blood cells causes the production of irregular red blood cells. Currently AML is treated by chemotherapy or bone marrow transplantation. However chemotherapy has several toxic effects to patients.

Despite the advances made in the art for treatment of cancer, particularly AML, there is still a need in the art for improved compositions useful for the treatment of cancer. The present invention fulfills this need.

### SUMMARY OF THE INVENTION

In one embodiment, the invention provides an isolated peptide that interacts with death receptor-6 (DR6) and is capable of modulating DR6 activity, wherein the peptide comprises a sequence selected from the group consisting of SEQ ID NO: 1, a fragment of SEQ ID NO: 1, a variant of SEQ ID NO: 1, SEQ ID NO: 2, a fragment of SEQ ID NO: 2, a variant of SEQ ID NO: 2, and any combination thereof.

In one embodiment, the peptide variant of SEQ ID NO: 1 is at least about 75% homologous to SEQ ID NO: 1.

In one embodiment, the peptide variant of SEQ ID NO: 2 is at least about 75% homologous to SEQ ID NO: 2.

The invention also provides a method for modulating activity of death receptor-6 (DR6) on a cell. In one embodiment, the method comprises contacting a cell with a peptide that interacts with DR6 and is capable of modulating DR6 activity, wherein the peptide comprises a sequence selected from the group consisting of SEQ ID NO: 1, a fragment of SEQ ID NO: 1, a variant of SEQ ID NO: 1, SEQ ID NO: 2, a fragment of SEQ ID NO: 2, a variant of SEQ ID NO: 2, and any combination thereof.

The invention also provides a method of treating or preventing a disease or condition in a subject in need thereof, wherein the disease or condition is associated with DR6 activity. In one embodiment, the method comprises administering to the subject a therapeutically effective amount of a composition comprising a peptide that interacts with DR6 and is capable of modulating activating DR6 activity, wherein the peptide comprises a sequence selected from the group consisting of SEQ ID NO: 1, a fragment of SEQ ID NO: 1, a variant of SEQ ID NO: 1, SEQ ID NO: 2, a fragment of SEQ ID NO: 2, a variant of SEQ ID NO: 2, and any combination thereof, whereby administration of the composition to the subject treats or prevents the disease or condition in the subject.

In one embodiment, the disease or condition that is associated with DR6 activity is cancer. In one embodiment, the cancer is selected from the group consisting of leukemia, glioma, breast cancer, melanoma, and any combination thereof.

The invention provides a method of detecting DR6. In one embodiment, the method comprises contacting DR6 with a peptide wherein the peptide comprises a sequence selected from the group consisting of SEQ ID NO: 1, a fragment of SEQ ID NO: 1, a variant of SEQ ID NO: 1, SEQ ID NO: 2, a fragment of SEQ ID NO: 2, a variant of SEQ ID NO: 2, and any combination thereof.

The invention also provides a kit for detecting DR6. In one embodiment, the kit comprises an instruction manual and a peptide, wherein the peptide comprises a sequence selected from the group consisting of SEQ ID NO: 1, a fragment of SEQ ID NO: 1, a variant of SEQ ID NO: 1, SEQ ID NO: 2, a fragment of SEQ ID NO: 2, a variant of SEQ ID NO: 2, and any combination thereof.

The invention also provides a method of diagnosing a disease or condition associated with DR6 activity in a subject. In one embodiment, the method comprises contacting a biological sample derived from a subject with a peptide, wherein the peptide comprises a sequence selected from the group consisting of SEQ ID NO: 1, a fragment of SEQ ID NO: 1, a variant of SEQ ID NO: 1, SEQ ID NO: 2, a fragment of SEQ ID NO: 2, a variant of SEQ ID NO: 2, and any combination thereof for a time sufficient to generate a DR6-peptide complex, wherein detection of the presence of the DR6-peptide complex diagnoses the subject with a disease or condition associated with DR6 activity.

The invention also provides an antibody that specifically binds to a peptide, wherein the peptide comprises a sequence selected from the group consisting of SEQ ID NO: 1, a fragment of SEQ ID NO: 1, a variant of SEQ ID NO: 1, SEQ ID NO: 2, a fragment of SEQ ID NO: 2, a variant of SEQ ID NO: 2, and any combination thereof.

The invention also provides an antibody that binds DR6 and prevents interaction with angiocidin or angiocidin-derived peptides. In one embodiment, the antibody specifically binds to a peptide comprising a sequence selected from the group consisting of SEQ ID NO: 1, a fragment of SEQ ID NO: 1, a variant of SEQ ID NO: 1, SEQ ID NO: 2, a fragment of SEQ ID NO: 2, a variant of SEQ ID NO: 2, and any combination thereof.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is an image showing that anti-DR6 antibody blocks the binding of Alex flour 488 labeled angiocidin to THP-1 cells.

FIG. 2 is an image showing that angiocidin binds the extracellular domain of DR6 (DR6 extracellular domain-FC chimera) in vitro.

FIG. 3 is an image showing that anti-DR6 antibody, unlabeled angiocidin, and 5 mer (ALKHR peptide) block binding of biotinylated angiocidin to the extracellular domain of DR6.

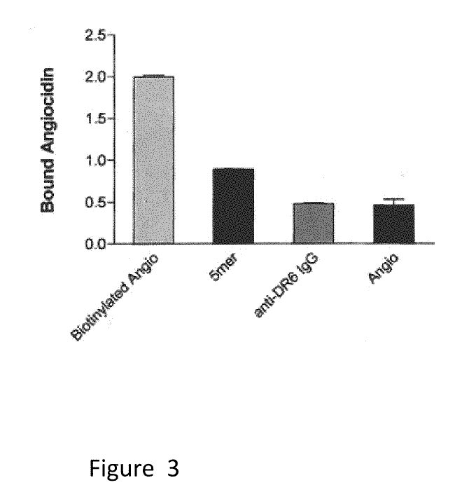

FIG. 4 is an image showing that anti-DR6 antibody blocks binding of angiocidin to DR6 in whole extracts of THP-1 cells.

FIG. 5 is an image showing that anti-DR6 antibody blocked angiocidin-mediated up-regulation of CD14 in THP-1 cells.

FIG. 6 is an image showing that THP-1 cells whose DR6 receptor has been stably down-regulated with short hairpin RNA against DR6 exhibited 61% inhibition of CD 14 up-regulation by angiocidin as compared to cells transfected with non-silencing control shRNA and exhibited 26% inhibition of CD54 up-regulation by angiocidin as compared to cells transfected with non-silencing control shRNA.

FIG. 7 is an image demonstrating that Angio-pep (ALKHR; SEQ ID NO: 1) a-peptide present in the anti-tumor domain of angiocidin (F₈₆ CTGIRVAHLALKHRQGKNH₁₅₀; SEQ ID NO: 2) mimics the differentiation activity of angiocidin.

FIG. 8 is an image demonstrating that angiocidin and its 5 mer active site peptide upregulate CD54 in THP-1 cells after 24 hours incubation whereas the reverse sequence 5 mer (r5 mer) is inactive.

FIG. 9 is an image demonstrating that angiocidin and the 5 mer peptide upregulate similar panels of cytokines in three leukemia cell lines.

FIG. 10 is an image showing the comparison between angiocidin, the 5 mer, and a 20 mer (F₈₆-H₁₀₅) FCTGIRVAHLALKHRQGKNH; SEQ ID NO: 2 on the expression of CD14 and CD54. Briefly, THP-1 cells were grown in the presence of various concentrations of peptide overnight and then the cells were assayed by flow cytometry for expression of CD14 and CD54.

FIG. 11 is an image showing the comparison between the 20 mer, 5 mer and angiocidin on the expression of CD14 and CD54 up-regulation in THP-1 cells and two primary leukemia cells. Cells were treated with peptide at 10 ug/ml for 12 hours and CD14 and CD54 were measured by flow cytometry.

### DETAILED DESCRIPTION OF THE INVENTION

The present invention is based on the discovery that the DR6 binding domain of angiocidin is located in a 5 amino acid sequence (ALKHR; SEQ ID NO: 1; also referred to as 5 mer) present near the N-terminal domain of angiocidin. Accordingly, in one embodiment, the invention includes compositions and methods of treating cancer by inducing the cellular differentiation activity of angiocidin using a peptide comprising SEQ ID NO: 1 or a derivative or variant thereof. In another embodiment, the invention includes compositions and methods of treating cancer by inducing the cellular differentiation activity of angiocidin using a peptide comprising SEQ ID NO: 2 or a derivative or variant thereof.

In one embodiment, the present invention provides peptide analogs of a 5 residue peptide from angiocidin that retains various desirable therapeutic properties and modifications and/or changes to the sequence having enhanced therapeutic benefits. Accordingly, the invention provides compositions comprising functional peptide analogs of angiocidin and uses thereof.

The invention is also based on the discovery that DR6 plays a role in the differentiation of leukemic cells. For example, blocking DR6 on THP-1 leukemic cells prevents angiocidin from inducing differentiation of leukemic cells. Accordingly, in one embodiment, the invention includes compositions and methods of treating cancer by inducing the cellular differentiation of a cancer cell using a DR6 agonist.

In another embodiment the invention includes a method of diagnosing a disease or condition associated with DR6 activity in a subject. In one embodiment, the method comprises contacting a biological sample derived from a subject with a peptide of the invention for a time sufficient to generate a DR6-peptide complex, wherein detection of the presence of the DR6-peptide complex diagnoses the subject with a disease or condition associated with DR6 activity.

### DEFINITIONS

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “agonist” in reference to a polypeptide is used in the broadest sense and includes any molecule that induces or increases the expression of a polypeptide encoding polynucleotide or induces or increases the stability and/or biological activity of a polypeptide. An agonist may include for example, small molecules, naturally occurring ligand agonists, polypeptide ligand agonists, and antibodies specific for an epitope of the polypeptide.

The term “amino acid sequence variant” refers to polypeptides having amino acid sequences that differ to some extent from a native sequence polypeptide. Ordinarily, amino acid sequence variants will possess at least about 70% homology, or at least about 80%, or at least about 90% homology to the native polypeptide. The amino acid sequence variants possess substitutions, deletions, and/or insertions at certain positions within the amino acid sequence of the native amino acid sequence.

As used herein, the terms “antibody” and “antibodies” (immunoglobulins) encompass monoclonal antibodies (including full-length monoclonal antibodies), polyclonal antibodies, multispecific antibodies (e.g., bispecific antibodies) formed from at least two intact antibodies, human antibodies, humanized antibodies, camelid antibodies, chimeric antibodies, single-chain Fvs (scFv), single-chain antibodies, single domain antibodies, domain antibodies, Fab fragments, F(ab′)2 fragments, antibody fragments that exhibit the desired biological activity, disulphide-linked Fvs (sdFv), and anti-idiotypic (anti-Id) antibodies (including, e.g., anti-Id antibodies to antibodies of the disclosure), intrabodies, and epitope-binding fragments of any of the above. In particular, antibodies include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer, AML, and the like.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the terms “conservative variation” or “conservative substitution” as used herein refers to the replacement of an amino acid residue by another, biologically similar residue. Conservative variations or substitutions are not likely to substantially change the shape and/or activity of the peptide chain. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Instructional material,” as that term is used herein, includes a publication, a recording, a diagram, or any other medium of expression which can be used to communicate the usefulness of the nucleic acid, peptide, and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a cell or a tissue of a subject. The instructional material of the kit may, for example, be affixed to a container that contains the nucleic acid, peptide, and/or compound of the invention or be shipped together with a container that contains the nucleic acid, peptide, and/or compound. Alternatively, the instructional material may be shipped separately from the container with the intention that the recipient uses the instructional material and the compound cooperatively.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “label” when used herein refers to a detectable compound or composition that is conjugated directly or indirectly to a probe to generate a “labeled” probe. The label may be detectable by itself (e.g. radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

By the term “modulating,” as used herein, is meant mediating a detectable increase or decrease in the level of a mRNA, polypeptide, or a response in a subject compared with the level of a mRNA, polypeptide or a response in the subject in the absence of a treatment or compound, and/or compared with the level of a mRNA, polypeptide, or a response in an otherwise identical but untreated subject. The term encompasses perturbing and/or affecting a native signal or response thereby mediating a beneficial therapeutic response in a subject, preferably, a human.

A “nucleic acid” refers to a polynucleotide and includes poly-ribonucleotides and poly-deoxyribonucleotides. Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, a “peptidomimetic” is a compound containing non-peptidic structural elements that is capable of mimicking the biological action of a parent peptide. A peptidomimetic may or may not comprise peptide bonds.

As used herein, “polynucleotide” includes cDNA, RNA, DNA/RNA hybrid, antisense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

By the term “specifically bind” or “specifically binds,” as used herein, is meant that a first molecule preferentially binds to a second molecule, but does not necessarily bind only to that second molecule.

As used herein, the term “substantially the same” amino acid sequence is defined as a sequence with at least 70%, preferably at least about 80%, more preferably at least about 85%, more preferably at least about 90%, even more preferably at least about 95%, and most preferably at least 99% homology with another amino acid sequence, as determined by the FASTA search method in accordance with Pearson & Lipman, 1988, Proc. Natl. Inst. Acad. Sci. USA 85:2444-48.

As used herein, the terms “therapy” or “therapeutic regimen” refer to those activities taken to alleviate or alter a disorder or disease state, e.g., a course of treatment intended to reduce or eliminate at least one sign or symptom of a disease or disorder using pharmacological, surgical, dietary and/or other techniques. A therapeutic regimen may include a prescribed dosage of one or more drugs or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease conditions, etc.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to compositions and methods of inducing differentiation of cancer cells, preferably leukemia cells. The methods generally comprise contacting the leukemia cell with an effective amount of a DR6 binding domain of angiocidin to induce differentiation and thereby inhibit the proliferative property of the cancer cell. In one embodiment, the DR6 binding domain of angiocidin is a peptide comprising the sequence of ALKHR (SEQ ID NO: 1). Accordingly, the invention includes peptides having the sequence of SEQ ID NO: 1 as well as mutants and variants thereof whereby the peptides mimic the differentiation activity of angiocidin. In another embodiment, the invention includes peptides having the sequence of SEQ ID NO: 2 as well as mutants and variants thereof whereby the peptides mimic the differentiation activity of angiocidin.

The invention provides peptide analogs of angiocidin useful for a wide variety of applications. The invention also provides methods of using the peptides as well as kits containing the peptides. The small peptides of the invention advantageously allow the relative ease of crossing tissue barriers, of providing active doses, of ease of synthesis in reproducibly pure large-scale quantities, and in some instances fewer side effects, compared to full length angiocidin.

In another embodiment, the invention provides methods of using the compositions of the invention in any therapeutic or prophylactic treatment known in the art, or subsequently discovered, that have been used with full-length angiocidin.

In another embodiment, the invention provides compositions and methods for treating cancer using a DR6 agonist.

In another embodiment, the peptides of the invention can be used as a reagent to bind DR6. For example, the peptides of the invention can be used as a diagnostics tool as a marker for the presence of DR6. In another embodiment, the invention provides composition and methods for detecting the presence of DR6 using a 5 mer comprising the amino acid sequence ALKHR; SEQ ID NO: 1. In another embodiment, the invention provides composition and methods for detecting the presence of DR6 using a 20 mer comprising the amino acid sequence set forth in SEQ ID NO: 2.

### Compositions

The invention includes a peptide comprising the sequence of (SEQ ID NO: 1), referred elsewhere herein as Angio-pep, as wells as mutants and derivatives thereof so long as the peptides bind to DR6. In one embodiment, the Angio-pep (SEQ ID NO: 1) of the invention includes variants of SEQ ID NO: 1, provided that the peptide still binds to DR6 and mimics the differentiation activity of angiocidin. In another embodiment, the invention includes variants of SEQ ID NO: 2, provided that the peptide still binds to DR6 and mimics the differentiation activity of angiocidin.

Variants may have mutations comprising insertions, deletions, or substitutions of amino acids. Variants preferably comprise conservative amino acid substitutions.

In one embodiment, the invention includes variants of the peptides of the invention. In one embodiment, variants differ from naturally-occurring peptides by conservative amino acid sequence differences or by modifications which do not affect sequence, or by both. For example, conservative amino acid changes may be made, which although they alter the primary sequence of the peptide, do not normally alter its function. Conservative amino acid substitutions typically include substitutions within the following groups:

glycine, alanine;

valine, isoleucine, leucine;

aspartic acid, glutamic acid;

asparagine, glutamine;

serine, threonine;

lysine, arginine;

phenylalanine, tyrosine.

In one embodiment, the peptide of the invention comprises a peptide having at least 75% homology with SEQ ID NO: 1 or SEQ ID NO: 2. In one embodiment, the peptide of the invention comprises a peptide having at least 80% homology with SEQ ID NO: 1 or SEQ ID NO: 2. In one embodiment, the peptide of the invention comprises a peptide having at least 85% homology with SEQ ID NO: 1 or SEQ ID NO: 2. In one embodiment, the peptide of the invention comprises a peptide having at least 90% homology with SEQ ID NO: 1 or SEQ ID NO: 2. In one embodiment, the peptide of the invention comprises a peptide having at least 95% homology with SEQ ID NO: 1 or SEQ ID NO: 2. In one embodiment, the peptide of the invention comprises a peptide having at least 99% homology with SEQ ID NO: 1 or SEQ ID NO: 2. In a further embodiment, the peptides of the invention comprise D-, L-, and unnatural isomers of amino acids.

In a further embodiment, the peptide of the invention comprise D-, L-, and unnatural isomers of amino acids. In one embodiment, the composition comprises a peptide comprising one or more unnatural or non-natural amino acids. Non-natural amino acids include, but are not limited to, the D-amino acids, 2,4-diaminobutyric acid, α-amino isobutyric acid, 2-aminoisobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, naphthalene, L-1-naphthalene, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general.

In one embodiment, the peptides of the invention may have mutations comprising insertions, deletions, or substitutions of amino acids. In one embodiment, the peptide of the invention comprises a peptide comprising about 3 to 10 amino acids and is at least about 80% homologous to SEQ ID NO: 1. In another embodiment, the peptide of the invention comprises a peptide comprising about 3 to 10 amino acids and is at least about 85% homologous to SEQ ID NO: 1. In another embodiment, the peptide of the invention comprises a peptide comprising about 3 to 10 amino acids and is at least about 90% homologous to SEQ ID NO: 1. In another embodiment, the peptide of the invention comprises a peptide comprising about 3 to 10 amino acids and is at least about 95% homologous to SEQ ID NO: 1. In another embodiment, the peptide of the invention comprises a peptide comprising about 3 to 10 amino acids and is at least about 99% homologous to SEQ ID NO: 1.

In one embodiment, the peptides of the invention may have mutations comprising insertions, deletions, or substitutions of amino acids. In one embodiment, the peptide of the invention comprises a peptide comprising about 10 to 100 amino acids and is at least about 80% homologous to SEQ ID NO: 2. In another embodiment, the peptide of the invention comprises a peptide comprising about 10 to 100 amino acids and is at least about 85% homologous to SEQ ID NO: 2. In another embodiment, the peptide of the invention comprises a peptide comprising about 10 to 100 amino acids and is at least about 90% homologous to SEQ ID NO: 2. In another embodiment, the peptide of the invention comprises a peptide comprising about 10 to 100 amino acids and is at least about 95% homologous to SEQ ID NO: 2. In another embodiment, the peptide of the invention comprises a peptide comprising about 10 to 100 amino acids and is at least about 99% homologous to SEQ ID NO: 2.

As known in the art the “similarity” between two peptides is determined by comparing the amino acid sequence and its conserved amino acid substitutes of one polypeptide to a sequence of a second peptide. Variants are defined to include polypeptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence and/or the ability to bind to ubiquitin or to a ubiquitylated protein. The present invention includes amino acid sequences that are at least 60%, 65%, 70%, 72%, 74%, 76%, 78%, 80%, 90%, or 95% similar or identical to the original amino acid sequence. The degree of identity between two polypeptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

Variants of suitable peptides of the invention can also be expressed. Variants may be made by, for example, the deletion, addition, or alteration of amino acids that have either (i) minimal influence on certain properties, secondary structure, and hydropathic nature of the polypeptide or (ii) substantial effect on one or more properties of the peptide mimetics of the invention.

Variants may also include, for example, a peptide conjugated to a linker or other sequence for ease of synthesis, purification, identification, or therapeutic use (i.e., delivery) of the peptide.

The variants of the peptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNA_{LYS}), could be modified with an amine specific photoaffinity label.

The peptides of the invention may be conjugated with other molecules, such as proteins, to prepare fusion proteins. This may be accomplished, for example, by the synthesis of N-terminal or C-terminal fusion proteins provided that the resulting fusion protein retains the functionality of the peptide of the invention.

Cyclic derivatives of the peptides the invention are also part of the present invention. Cyclization may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The peptides of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

Peptides of the invention may also have modifications. Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are peptides which have been modified using ordinary molecular biological techniques so as to improve their resistance to proteolytic degradation or to optimize solubility properties or to render them more suitable as a therapeutic agent. Such variants include those containing residues other than naturally-occurring L-amino acids, e.g., D-amino acids or non-naturally-occurring synthetic amino acids. The peptides of the invention may further be conjugated to non-amino acid moieties that are useful in their therapeutic application. In particular, moieties that improve the stability, biological half-life, water solubility, and/or immunologic characteristics of the peptide are useful. A non-limiting example of such a moiety is polyethylene glycol (PEG).

Covalent attachment of biologically active compounds to water-soluble polymers is one method for alteration and control of biodistribution, pharmacokinetics, and often, toxicity for these compounds (Duncan et al., 1984, Adv. Polym. Sci. 57:53-101). Many water-soluble polymers have been used to achieve these effects, such as poly(sialic acid), dextran, poly(N-(2-hydroxypropyl)methacrylamide) (PHPMA), poly(N-vinylpyrrolidone) (PVP), poly(vinyl alcohol) (PVA), poly(ethylene glycol-co-propylene glycol), poly(N-acryloyl morpholine (PAcM), and poly(ethylene glycol) (PEG) (Powell, 1980, Polyethylene glycol. In R. L. Davidson (Ed.) Handbook of Water Soluble Gums and Resins. McGraw-Hill, New York, chapter 18). PEG possess an ideal set of properties: very low toxicity (Pang, 1993, J. Am. Coll. Toxicol. 12: 429-456) excellent solubility in aqueous solution (Powell, supra), low immunogenicity and antigenicity (Dreborg et al., 1990, Crit. Rev. Ther. Drug Carrier Syst. 6: 315-365). PEG-conjugated or “PEGylated” protein therapeutics, containing single or multiple chains of polyethylene glycol on the protein, have been described in the scientific literature (Clark et al., 1996, J. Biol. Chem. 271: 21969-21977; Hershfield, 1997, Biochemistry and immunology of poly(ethylene glycol)-modified adenosine deaminase (PEG-ADA). In J. M. Harris and S. Zalipsky (Eds) Poly(ethylene glycol): Chemistry and Biological Applications. American Chemical Society, Washington, D.C., p 145-154; Olson et al., 1997, Preparation and characterization of poly(ethylene glycol)ylated human growth hormone antagonist. In J. M. Harris and S. Zalipsky (Eds) Poly(ethylene glycol): Chemistry and Biological Applications. American Chemical Society, Washington, D.C., p 170-181).

A peptide of the invention may be synthesized by conventional techniques. For example, the peptides of the invention may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis.)

The peptides may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the alpha-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method which utilized tert-butyloxcarbonyl as the alpha-amino protecting group, and the FMOC method which utilizes 9-fluorenylmethyloxcarbonyl to protect the alpha-amino of the amino acid residues, both which methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function, e.g. with DCC, can then proceed by addition of the desired alcohol, followed by de-protection and isolation of the esterified peptide product.

In one embodiment, the peptides of the invention are manufactured by solid phase peptide synthesis using Fmoc chemistry. In certain embodiments, after synthesis the Fmoc group is deprotected at the N-terminus, the side chain protection group is deprotected, and the peptide is cleaved from the resin. In one embodiment, the resin is a Cl-resin. In one embodiment, the condensation reaction reagent is DIC+HOBT. In one embodiment deprotection is done using Pip. In certain embodiments, the synthesized peptides are purified by RP-HPLC using a solvent of acetonitrile+deionized with TFA as the buffer. In one embodiment, the peptides are purified by gradient elution.

In other embodiments, the subject peptide therapeutic agents are peptidomimetics of the peptide modulators. Peptidomimetics are compounds based on, or derived from, peptides and proteins. The peptidomimetics of the present invention typically can be obtained by structural modification of a known peptide modulator sequence using unnatural amino acids, conformational restraints, isosteric replacement, and the like. The subject peptidomimetics constitute the continuum of structural space between peptides and non-peptide synthetic structures; peptidomimetics may be useful, therefore, in delineating pharmacophores and in helping to translate peptides into nonpeptide compounds with the activity of the parent peptide inhibitors.

Moreover, as is apparent from the present disclosure, mimetopes of the subject peptide modulators can be provided. Such peptidomimetics can have such attributes as being non-hydrolyzable (e.g., increased stability against proteases or other physiological conditions which degrade the corresponding peptide), increased specificity and/or potency, and increased cell permeability for intracellular localization of the peptidomimetic. For illustrative purposes, peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988, p 123), C-7 mimics (Huffman et al. in Peptides: Chemistry and Biologyy, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), β-aminoalcohols (Gordon et al. (1985) Biochem Biophys Res Commun 126:419; and Dann et al. (1986) Biochem Biophys Res Commun 134:71), diaminoketones (Natarajan et al. (1984) Biochem Biophys Res Commun 124:141), and methyleneamino-modified (Roark et al. in Peptides: Chemistry and Biology, G. R. Marshall ed., ESCOM Publisher: Leiden, Netherlands, 1988, p 134). Also, see generally, Session III: Analytic and synthetic methods, in Peptides: Chemistry and Biology, G. R. Marshall ed., ESCOM Publisher: Leiden, Netherlands, 1988).

In addition to a variety of side chain replacements which can be carried out to generate the peptidomimetics, the present invention specifically contemplates the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methyleneamino, (iv) phosphonamides, and (v) sulfonamides.

The peptides of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

### Nucleic Acid

Included in the invention are nucleic acid sequences that encode the peptides of the invention. In one embodiment, the invention includes nucleic acid sequences corresponding to the amino acid sequence of SEQ ID NO: 1. In another embodiment, the invention includes nucleic acid sequences corresponding to the amino acid sequence of SEQ ID NO: 2. Accordingly, subclones of a nucleic acid sequence encoding a peptide mimetic of the invention can be produced using conventional molecular genetic manipulation for subcloning gene fragments, such as described by Sambrook et al., Molecular Cloning: A Laboratory Manual, Cold Springs Laboratory, Cold Springs Harbor, New York (2012), and Ausubel et al. (ed.)

Biological preparation of a peptide of the invention involves expression of a nucleic acid encoding a desired peptide. An expression cassette comprising such a coding sequence may be used to produce a desired peptide for use in the method of the invention.

In the context of an expression vector, the vector can be readily introduced into a host cell, e.g., mammalian, bacterial, yeast or insect cell by any method in the art. Coding sequences for a desired peptide of the invention may be codon optimized based on the codon usage of the intended host cell in order to improve expression efficiency as demonstrated herein. Codon usage patterns can be found in the literature (Nakamura et al., 2000, Nuc Acids Res. 28:292). Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The expression vector can be transferred into a host cell by physical, biological or chemical means, discussed in detail elsewhere herein.

Examples of biological methods to prepare the peptides of the present invention may utilize methods provided in published US Patent application number US 2009/0069241, which is incorporated herein in its entirety.

To ensure that the peptide obtained from either chemical or biological synthetic techniques is the desired peptide, analysis of the peptide composition can be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

The isolated nucleic acid may comprise any type of nucleic acid, including, but not limited to DNA and RNA. For example, in one embodiment, the composition comprises an isolated DNA molecule, including for example, an isolated cDNA molecule, encoding a peptide of the invention, or functional fragment thereof. In one embodiment, the composition comprises an isolated RNA molecule encoding a peptide of the invention, or a functional fragment thereof. The isolated nucleic acids may be synthesized using any method known in the art.

The nucleic acid molecules of the present invention can be modified to improve stability in serum or in growth medium for cell cultures. Modifications can be added to enhance stability, functionality, and/or specificity and to minimize immunostimulatory properties of the nucleic acid molecule of the invention. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule.

In one embodiment of the present invention the nucleic acid molecule may contain at least one modified nucleotide analogue. For example, the ends may be stabilized by incorporating modified nucleotide analogues.

Non-limiting examples of nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In preferred backbone-modified ribonucleotides the phosphoester group connecting to adjacent ribonucleotides is replaced by a modified group, e.g., of phosphothioate group. In preferred sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In some instances, the nucleic acid molecule comprises at least one of the following chemical modifications: 2′-H, 2′-O-methyl, or 2′-OH modification of one or more nucleotides. In certain embodiments, a nucleic acid molecule of the invention can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance the nucleic acid molecules of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target.

In one embodiment, the nucleic acid molecule includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In one embodiment, the nucleic acid molecule includes at least one 2′-O-methyl-modified nucleotide, and in some embodiments, all of the nucleotides of the nucleic acid molecule include a 2′-O-methyl modification.

Nucleic acid agents discussed herein include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, preferably as occur naturally in the human body. The art has referred to rare or unusual, but naturally occurring, RNAs as modified RNAs, see, e.g., Limbach et al. (Nucleic Acids Res., 1994, 22:2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, preferably different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone.

Modifications of the nucleic acid of the invention may be present at one or more of, a phosphate group, a sugar group, backbone, N-terminus, C-terminus, or nucleobase.

### Methods

In various embodiments, the present invention provides a method of treating cancer. In one embodiment, the method includes the use of a peptide of the invention to treat a cancer that is treatable with angiocidin. The methods generally comprise administering to a subject in need thereof an amount of a peptide of the invention to treat the cancer. The peptide of the invention can be administered to the subject as a composition comprising a pharmaceutically acceptable carrier. The amount of the peptide of the invention administered to the subject can vary according to the type of tumor, or other variables.

In another embodiment, the method includes the use of a DR6 agonist to treat a cancer that is treatable with angiocidin. The methods generally comprise administering to a subject in need thereof an amount of a DR6 agonist to treat the cancer. The DR6 agonist can be administered to the subject as a composition comprising a pharmaceutically acceptable carrier. The amount of the DR6 agonist administered to the subject can vary according to the type of tumor, or other variables.

The invention also provides methods for arresting the growth of a tumor cell. In one embodiment, the methods generally comprise contacting the tumor cell with an amount of a peptide of the invention effective to arrest growth of the tumor cell. In another embodiment, the methods generally comprise contacting the tumor cell with an amount of a DR6 agonist of the invention effective to arrest growth of the tumor cell. In yet another embodiment, the methods generally comprise contacting the tumor cell with an amount of a peptide and a DR6 agonist of the invention effective to arrest growth of the tumor cell. Accordingly, the methods generally comprise contacting the tumor cell with a peptide of the invention, a DR6 agonist of the invention, or both a peptide and DR6 agonist of the invention to arrest growth of the tumor cell. In some aspects, the tumor cell will undergo apoptosis subsequent to its exposure to a peptide of the invention, a DR6 agonist of the invention, or both a peptide and DR6 agonist of the invention, and the apoptosis may begin subsequent to the growth arrest. The peptide of the invention, DR6 agonist of the invention, or both the peptide and DR6 agonist of the invention can be used to arrest the growth of any tumor cell, with glioma cells, breast cancer cells, leukemia cells, and melanoma cells being highly preferred.

The invention also provides methods for inducing differentiation of a leukemia cell. The methods generally comprise contacting the leukemia cell with an amount of a peptide of the invention, a DR6 agonist of the invention, or both a peptide and DR6 agonist of the invention to induce differentiation of the leukemia cell. The differentiation of the leukemia cell induces genotypic and phenotypic changes that inhibit, and that are capable of reversing the tumorigenicity of the leukemia cell, for example, making the leukemia cell less cancerous. For example, differentiation of leukemic cells may result in downregulation and expression of the oncogene. Cells can also be considered less cancerous if they fail to grow in soft agar and display a less invasive phenotype, or are capable of phagocytosis. In some aspects, the differentiation induces the leukemia cell to confer a non-cancerous phenotype, for example, a phenotype characterized by a cell capable of anchorage-dependent growth with a well-defined cytoskeleton. It is believed that the compositions of the invention may induce leukemic cells to lose their potential to initiate a tumor when engrafted into an immunocompromised host. The differentiated phenotype in leukemia can be a cell expressing molecules normally expressed by terminally differentiated immune cells such as CD14, a macrophage marker, and molecules that can present antigens to T cells. In addition, such phenotypes include that differentiated cells no longer can self-renew, have a limited lifespan, downregulate oncogenes, cannot metastasize, and are contact inhibited in vitro.

Further, one of skill in the art would appreciate, when equipped with this disclosure and the methods exemplified herein, that a peptide or DR6 agonist composition includes such peptides and DR6 agonist as discovered in the future, as can be identified by well-known criteria in the art of pharmacology, such as the physiological results as described in detail herein and/or as known in the art. Therefore, the present invention is not limited in any way to any particular peptide and DR6 agonist composition as exemplified or disclosed herein; rather, the invention encompasses those peptide and DR6 compositions that would be understood by the routineer to be useful as are known in the art and as are discovered in the future.

A method of treating a human or other mammal in need thereof with a peptide of the invention, a DR6 agonist of the invention, or both a peptide and DR6 agonist of the invention comprising administering to the human or other mammal a therapeutically effective dose of a peptide of the invention, a DR6 agonist of the invention, or both a peptide and DR6 agonist of the invention is also provided. A method for activating or inducing the differentiation of native stem cells comprising administering a peptide of the invention, a DR6 agonist of the invention, or both a peptide and DR6 agonist of the invention to one or more native stem cells, wherein the stem cells are activated or induced to differentiate into lineages of the three germ layers. A method for inducing differentiation of tumor-derived stem cells comprising administering a peptide of the invention, a DR6 agonist of the invention, or both a peptide and DR6 agonist of the invention to tumor-derived stem cells in vivo or in vitro, wherein the tumor-derived cancer stem cells are induced to differentiate into cells which lose their ability to hyperproliferate and possess a gene expression profile of terminally differentiated cells, similar to normal cells encompassing the tumor.

Compositions of the invention may be administered to a subject by any appropriate means, such as enteral, parenteral, transdermal, or by direct injection or application to a diseased site.

### Antibodies

In one embodiment, the peptides of the present invention may be used for the generation of an antibody. For example, one or more peptides of the invention may be used to generate an antibody that specifically binds to the peptide and therefore also to a region of angiocidin.

Methods of making and using antibodies are well known in the art. For example, polyclonal antibodies are generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. In the present invention, the peptides of the invention may serve as the antigen. The chimeric proteins are produced by cloning the appropriate nucleic acids encoding the marker protein into a plasmid vector suitable for this purpose, such as but not limited to, pMAL-2 or pCMX.

The present invention should be construed to encompass antibodies which bind to the specific antigens of interest (i.e. peptide analogs of angiocidin), and are able to bind the antigen present on Western blots, in solution in enzyme linked immunoassays, in fluorescence activated cells sorting (FACS) assays, in magenetic-actived cell sorting (MACS) assays, immunocytochemistry, immunoprecipitation, and in immunofluorescence microscopy of a cell transiently transfected with a nucleic acid encoding at least a portion of the antigenic protein, for example.

The antibodies of the invention may be used to neutralize the activity or to inhibit the activity of angiocidin. For example, an antibody generated with the use of the peptides of the invention may be used as a therapeutic composition in order to inhibit the activity of angiocidin, in conditions where excessive angiocidin expression or activity is deleterious or associated with a particular disease or disorder.

The antibodies can be produced by immunizing an animal such as, but not limited to, a rabbit, a mouse or a camel, with an antigenic peptide of the invention, or a portion thereof, by immunizing an animal using a protein comprising at least a portion of the peptide, or a fusion protein including a tag polypeptide portion comprising, for example, a maltose binding protein tag polypeptide portion, covalently linked with a portion comprising the appropriate amino acid residues. One skilled in the art would appreciate, based upon the disclosure provided herein, that smaller fragments of these peptide can also be used to produce antibodies that specifically bind the antigen of interest.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

Further, the skilled artisan, based upon the disclosure provided herein, would appreciate that using a non-conserved immunogenic portion can produce antibodies specific for the non-conserved region thereby producing antibodies that do not cross-react with other proteins which can share one or more conserved portions. Thus, one skilled in the art would appreciate, based upon the disclosure provided herein, that the non-conserved regions of an antigen of interest can be used to produce antibodies that are specific only for that antigenic peptide and do not cross-react non-specifically with other proteins or peptides, including other types of angiocidin peptide fragments.

The invention encompasses monoclonal, synthetic antibodies, and the like. One skilled in the art would understand, based upon the disclosure provided herein, that the crucial feature of the antibody of the invention is that the antibody bind specifically with an antigen of interest. That is, the antibody of the invention recognizes an antigen of interest or a fragment thereof (e.g., an immunogenic portion or antigenic determinant thereof), on Western blots, in immunostaining of cells, and immunoprecipitates the antigen using standard methods well-known in the art.

One skilled in the art would appreciate, based upon the disclosure provided herein, that the antibodies can be used to immunoprecipitate and/or immuno-affinity purify their cognate antigen as described in detail elsewhere herein, and additionally, by using methods well-known in the art.

The skilled artisan would appreciate, based upon the disclosure provided herein, that that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

In one embodiment of the invention, a phage antibody library may be generated, as described in detail elsewhere herein. To generate a phage antibody library, a cDNA library is first obtained from mRNA which is isolated from cells, e.g., peripheral blood lymphocytes, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al., supra.

Bacteriophage which encode the desired antibody, may be engineered such that the protein is displayed on the surface thereof in such a manner that it is available for binding to its corresponding binding peptide, e.g., the antigen against which the antibody is directed, such as an antigen of interest (i.e. the angiocidin peptide fragments of the invention). Thus, when bacteriophage which express a specific antibody are incubated in the presence of the corresponding antigen, the bacteriophage will bind to the antigen. Bacteriophage which do not express the antibody will not bind to the antigen. Such panning techniques are well known in the art and are described for example, in Wright et al. (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al. (1991, J. Mol. Biol. 222:581-597). Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Biol. 248:97-105). In another embodiment of the invention, phage-cloned antibodies derived from immunized animals can be humanized by known techniques.

One of skill in the art will further appreciate that the present invention encompasses the use of antibodies derived from camelid species. That is, the present invention includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies. Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The production of polyclonal sera from camelid species is substantively similar to the production of polyclonal sera from other animals such as sheep, donkeys, goats, horses, mice, chickens, rats, and the like. The skilled artisan, when equipped with the present disclosure and the methods detailed herein, can prepare high-titers of antibodies from a camelid species. As an example, the production of antibodies in mammals is detailed in such references as Harlow et al., (1998, Antibodies: A Laboratory Manual, Cold Spring Harbor, New York).

### Pharmaceutical

A peptide of the invention, a DR6 agonist of the invention, or both a peptide and DR6 agonist of the invention can be formulated and administered to a subject, as now described. The invention encompasses the preparation and use of pharmaceutical compositions comprising a composition useful for the treatment of a disease or disorder, including, but not limited to cancer. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. As used herein, the term “pharmaceutically-acceptable carrier” means a chemical composition with which an appropriate peptide composition, may be combined and which, following the combination, can be used to administer the appropriate composition to a subject.

The pharmaceutical compositions useful for practicing the invention may be administered to deliver a dose of between about 0.1 ng/kg/day and 100 mg/kg/day. In various embodiments, the pharmaceutical compositions useful in the methods of the invention may be administered, by way of example, systemically, parenterally, orally, or topically. In addition to the appropriate therapeutic composition, such pharmaceutical compositions may contain pharmaceutically acceptable carriers and other ingredients known to enhance and facilitate drug administration.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Pharmaceutical compositions that are useful in the methods of the invention may be prepared, packaged, or sold in formulations suitable for oral, parenteral, topical, intravenous, intramuscular, and other known routes of administration.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, cutaneous, subcutaneous, intraperitoneal, intravenous, and intramuscular.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g., sterile pyrogen-free water) prior to parenteral administration of the reconstituted composition.

Typically dosages of the compound of the invention which may be administered to an animal, preferably a human, range in amount from about 0.01 mg to 20 about 100 g per kilogram of body weight of the animal. While the precise dosage administered will vary depending upon any number of factors, including, but not limited to, the type of animal and type of disease state being treated, the age of the animal and the route of administration. Preferably, the dosage of the compound will vary from about 1 mg to about 100 mg per kilogram of body weight of the animal. More preferably, the dosage will vary from about 1 μg to about 1 g per kilogram of body weight of the animal. The compound can be administered to an animal as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

Numerous vectors and other compositions and methods are well known for administering a peptide or a nucleic acid construct encoding a peptide to cells or tissues. Therefore, the invention includes a method of administering a peptide or a nucleic acid encoding a peptide that is an agonist of a GPCR. (Sambrook et al., 2001, Molecular Cloning: A Laboratory Manual, Cold Spring Harbor Laboratory, New York; Ausubel et al., 1997, Current Protocols in Molecular Biology, John Wiley & Sons, New York).

It will be appreciated by one of skill in the art, when armed with the present disclosure including the methods detailed herein, that the invention is not limited to treatment of a disease or disorder that is already established. Particularly, the disease or disorder need not have manifested to the point of detriment to the subject; indeed, the disease or disorder need not be detected in a subject before treatment is administered. That is, significant disease or disorder does not have to occur before the present invention may provide benefit. Therefore, the present invention includes a method for preventing a disease or disorder in a subject, in that a composition, as discussed elsewhere herein, can be administered to a subject prior to the onset of the disease or disorder, thereby preventing the disease or disorder. The preventive methods described herein also include the treatment of a subject that is in remission for the prevention of a recurrence of a disease or disorder.

### Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: DR6 is the Cell Surface Receptor on THP-1 Leukemia Cells that Mediates the Differentiating Activity of Angiocidin

Angiocidin is a peptide that has been shown to inhibit tumor growth, angiogenesis and promotes leukemic cell differentiation. Binding partners of angiocidin have been discovered to include integrins, extracellular matrix proteins and polyubiquitinated proteins that play roles in its anti-tumor activity. The results presented herein identify a new binding protein for an angiocidin that binds angiocidin more than 100 times more strongly than the other binding proteins and peptides that have previously been reported for angiocidin. The results presented herein demonstrate that death receptor-6 (DR6), a transmembrane receptor belonging to the tumor necrosis family of receptors, binds to angiocidin with heightened affinity.

Death receptor 6 (DR6) is a member of the second family of death receptors. DR6 is widely expressed, but appears to function differently in different cell types. DR6 mRNA has been observed in heart, brain, placental, pancreas, lymph node, thymus and prostate tissues. Lower levels have been observed in other cell types including skeletal muscle, kidney and testes, but little or no expression has previously been observed in adult liver or any lines of hematopoeitic origin. Interestingly, it has been observed that DR6 is capable of inducing apoptosis in only a subset of cells tested. For example, overexpression of DR6 in HeLa S3 cervical carcinoma cells resulted in apoptosis in a death-domain-dependent manner (Pan et al. FEBS 431:351-356 (1998)). In addition, Nikoleav et al. (Nature 457:981-990 (2009)) have shown that beta-amyloid precursor protein (APP) is a DR6 ligand and suggested that the binding of an APP fragment to DR6 triggers degeneration of neuronal cell bodies and axons. In contrast, DR6 did not induce cell death in MCF7 (a human breast adenocarcinoma line) cells (Pan et al. FEBS 431:351-356 (1998)).

The results presented herein also demonstrate that DR6 is expressed on THP-I leukemia cells, a cell line derived from acute myeloid leukemia (AML), and mediates the differentiating activity of angiocidin which transforms THP-I leukemia cells into normal macrophage-like cells. This conclusion is based on experiments which show that blocking DR6 on THP-I leukemia with either a dominant-negative mutant of DR6, an anti-DR6 antibody, or gene silencing DR6 vectors blocks the leukemia differentiating activity of angiocidin. Additionally, the DR6 binding domain of angiocidin is located in a 5 amino acid sequence (ALKHR; SEQ ID NO: 1) present near the N-terminal domain of angiocidin. Accordingly, the 5 mer (ALKHR; SEQ ID NO: 1) is a potentially non-toxic alternative treatment of AML

The results of the experiments are now described.

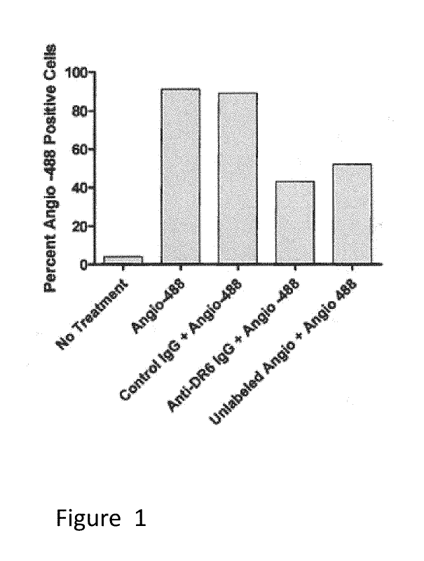

Experiments were performed to evaluate the interaction between DR-6 and angiocidin. Results showed that anti-DR6 antibody blocks the binding of Alexa Flour 488 labeled angiocidin to THP-1 cells (FIG. 1). Briefly, THP-1 cells were plated in 6 well dishes in serum-free media. Cells were untreated or treated for 30 min at 37° with either 2.5 μg/ml of Angiocidin labeled with Alexa Fluor 488 (Angio-488), Angio-448+10 μg/ml control rabbit IgG, Angio-488+10 μg/ml rabbit Anti-DR6 IgG, Angio-488+10 μg/ml unlabeled Angiocidin. Cells were washed twice in PBS and the percent positive Angio-488 measured by flow cytometry. It was observed that anti-DR6 IgG inhibited binding of Angiocidin to THP-1 cells. It was also observed that unlabeled angiocidin competed with the binding of Angio-488 indicating that the labeling of angiocidin with Alexa Fluor 488 did not alter its affinity for cell bound DR6.

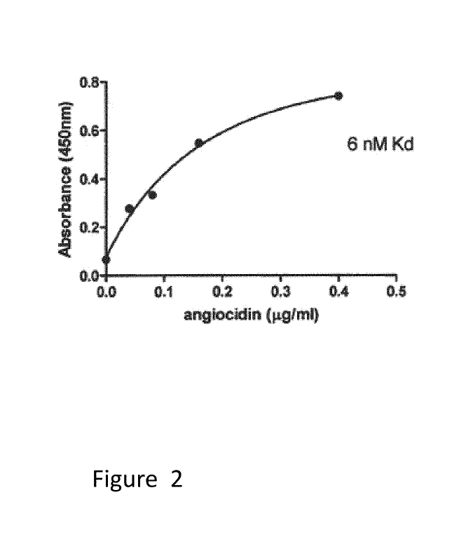

The next experiment was performed to characterize the binding of DR6 with angiocidin. Briefly, wells of a microtiter plate were coated with 100 ng of a DR6 extracellular-Fc chimeric recombinant protein overnight. Wells were then washed in PBS and blocked with 1% BSA. Blocked wells were incubated with shaking with 200 μl of PBS containing various concentrations of biotinylated angiocidin for one hour. Wells were than washed and incubated with shaking with a 1:10,000 dilution of a 1 mg/ml solution of streptavidin-HRP in PBS. Wells were washed 3 times in PBS and bound streptavidin HRP was detected by incubation of the wells with 100 μl of 3,3′,5,5′-tetramethylbenzidine (TMB) with shaking for 5 min. Reactions were stopped by the addition of 50 μl of 1 M HCl and the color read in an ELISA reader at 450 nm. It was observed that angiocidin binds the extracellular domain of DR6 (DR6 extracellular domain-FC chimera) in vitro (FIG. 2).

The next experiment was performed to further characterize the binding between DR6 and angiocidin. It was observed that the binding of angiocidin to DR6 was blocked with anti-DR6 IgG, 5 mer peptide, and unlabeled angiocidin. Wells contained either 1 μg/ml of biotinylated angiocidin alone or 50 μg/ml 5 mer, 10 μg/ml of anti-DR6 IgG or 50 μg/ml unlabeled angiocidin.

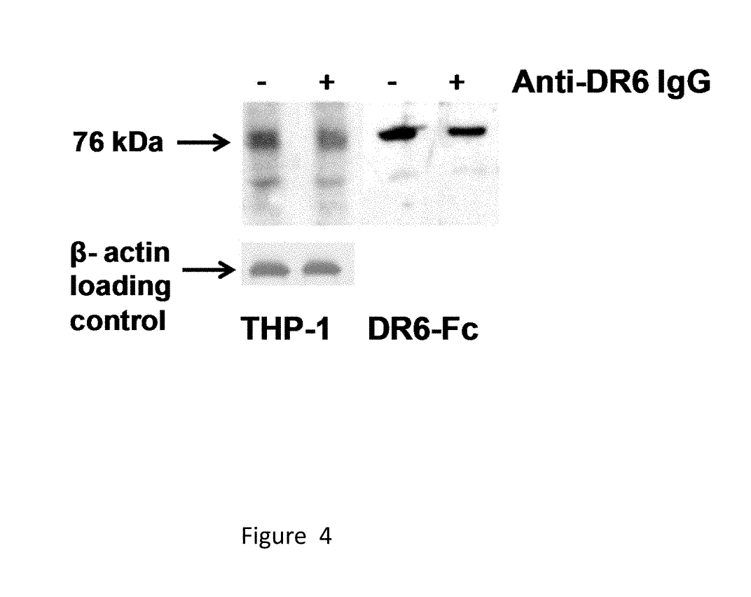

It was also observed that anti-DR6 antibody blocked binding of angiocidin to DR6 in whole extracts of THP-1 cells (FIG. 4). Briefly, SDS detergent extracts of THP-1 cells were separated on SDS-PAGE and blotted onto PVD membranes. Membranes were washed in tris-buffered saline containing 0.1% Tween 20 detergent (TBS-T) and 1% BSA and incubated with 0.1 mg/ml of biotinylated angiocidin in the presence or absence of 10 μg/ml rabbit Anti-DR6 antibody. After incubation for one hour membranes were washed in TBS-T and incubated with 1:10,000 dilution of a 1 mg/ml solution of streptavidin HRP in PBS for 30 min. Membranes were then washed, treated with a chemiluminescent substrate for HRP and developed for chemiluminescence using X-ray film. In the panel on the right, recombinant DR6-Fc was used as the positive control. It was observed that angiocidin binds to DR6 in whole extracts of THP-1 cells.

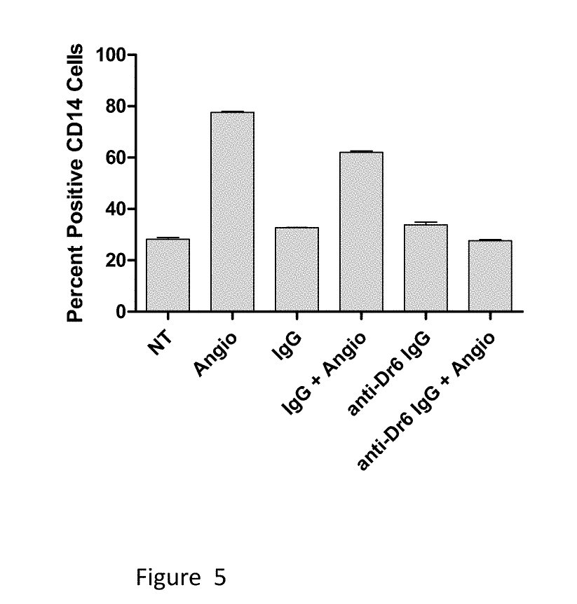

The results also demonstrate that anti-DR6 antibody blocked angiocidin-mediated up-regulation of CD14 in THP-1 cells (FIG. 5). Briefly, Anti-DR6 IgG and DR6-Fc inhibit the angiocidin-mediated upregulation of CD14 in THP-1 cells. THP-1 cells were grown for 24 hours in RPMI containing 1% fetal calf serum (NT) or containing the following: 2.5 μg/ml Angiocidin, 0.10 μg/ml DR6-Fc, 2.5 μg/ml Angiocidin plus 0.10 μg/ml, DR6-Fc, 10 μg/ml rabbit IgG, 2.5 μg/ml Angiocidin plus 10 μg/ml rabbit IgG, 10 μg/ml rabbit anti-DR6 IgG, 2.5 μg/ml Angiocidin plus 10 μg/ml rabbit anti-DR6 IgG. Percent CD14 positive cells were measured by flow cytometry.

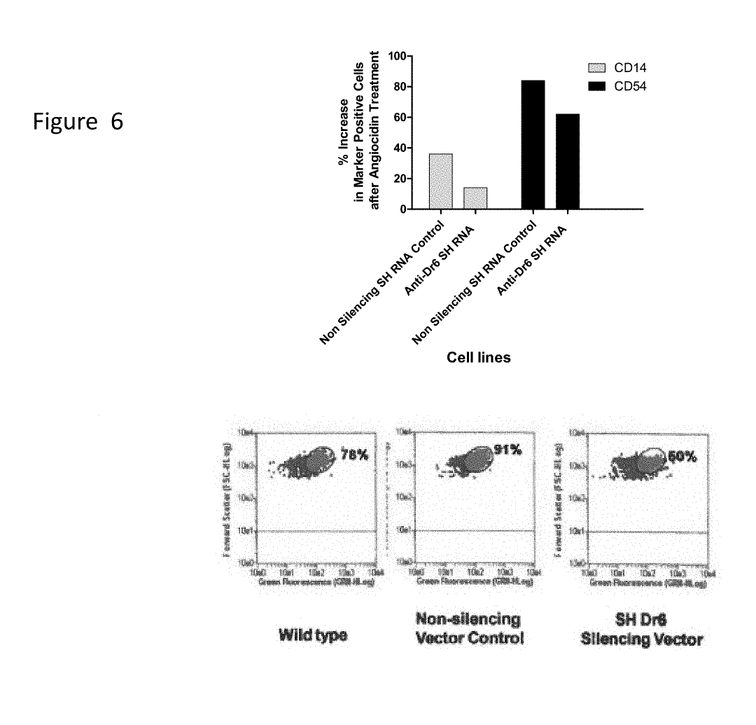

The results presented herein (FIG. 6) also show that THP-1 cells whose DR6 receptor has been stably down-regulated with short hairpin RNA against DR6 exhibited 61% inhibition of CD 14 up-regulation by angiocidin as compared to cells transfected with non-silencing control and exhibited 26% inhibition of CD54 up-regulation by angiocidin as compared to cells transfected with non-silencing control. Briefly, THP-1 cells expressing less DR6 receptor lose their capacity to up-regulate CD14 and CD54 in response to angiocidin. THP-1 cells were transduced with Lenti viruses expressing silencing anti-DR6 short hairpin RNA or non-silencing RNA. Transduced cells were isolated in puromycin containing media. Panel on right shows the percent of cells expressing DR6. Cells were then cultured for 24 hours in media with no additions or media containing 5 μg/ml angiocidin. Percent increase in marker CD 14 and CD54 expression was measured by flow cytometry.

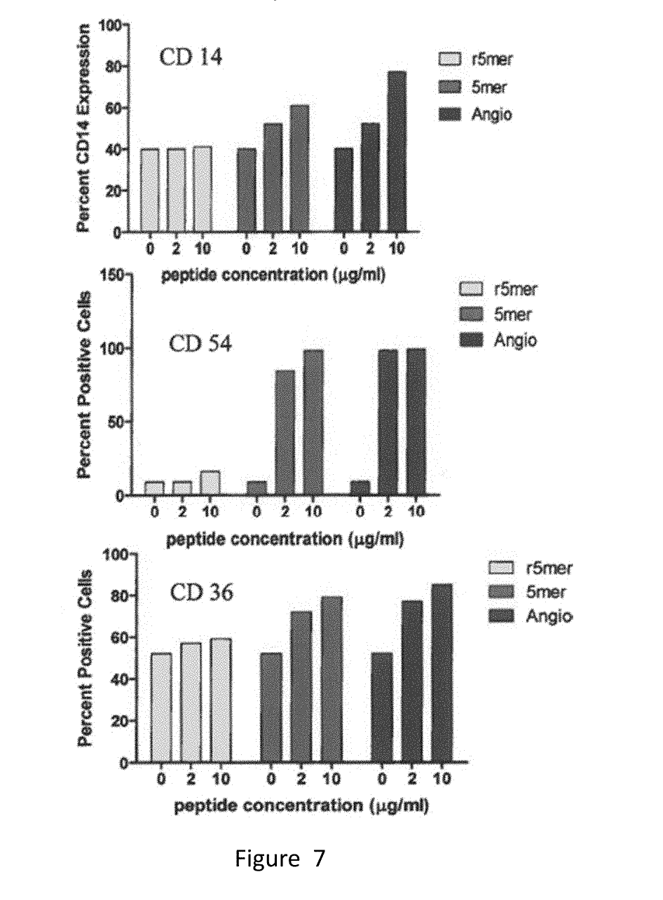

The results presented here (FIG. 7) demonstrate that Angio-pep (ALKHR), a-peptide present in the anti-tumor domain of angiocidin (F₈₆ CTGIRVAHLALKHRQGKNH₁₀₅), mimics the differentiation activity of angiocidin. The 5 mer peptide mimics the differentiation activity of angiocidin as measured by upregulation of CD14, CD54, CD36. THP-1 cells were cultured in RPMI media for 24 hours containing no peptides or angiocidin or 2, 10 μg/ml of either angiocidin, 5 mer, peptide or a control peptide, r5 mer having the reverse sequence of the 5 mer. Marker expression was measured by flow cytometry.

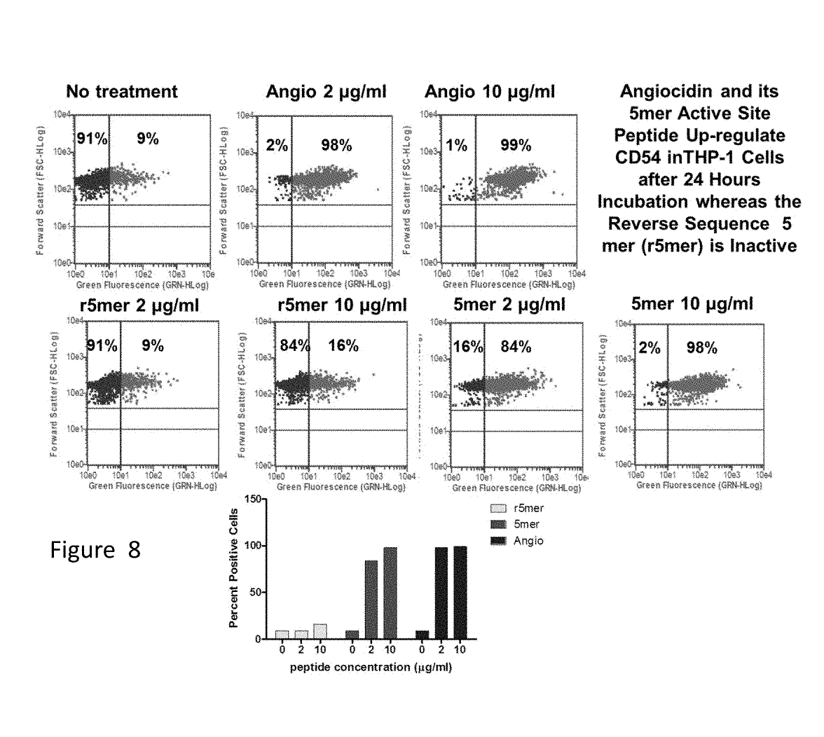

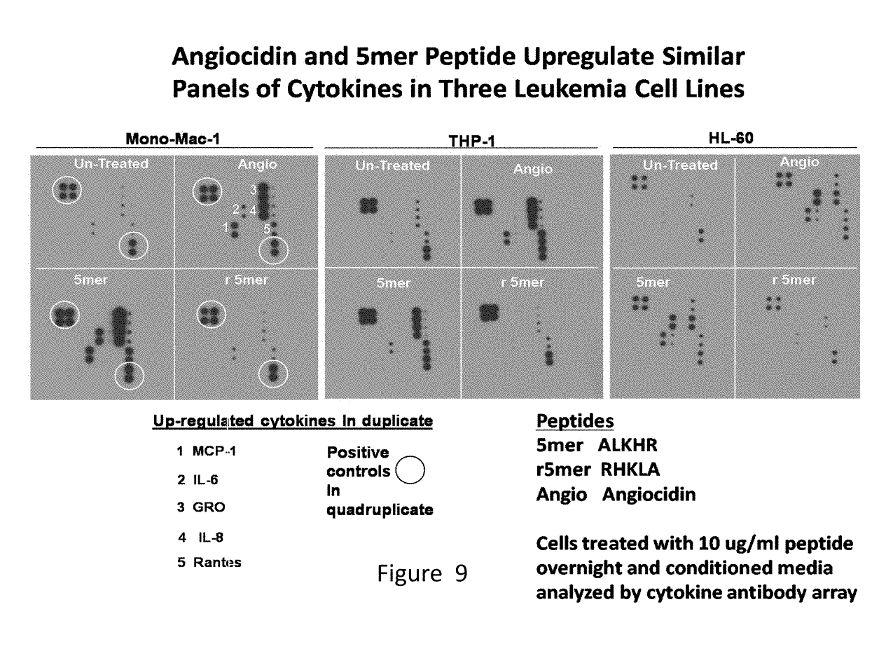

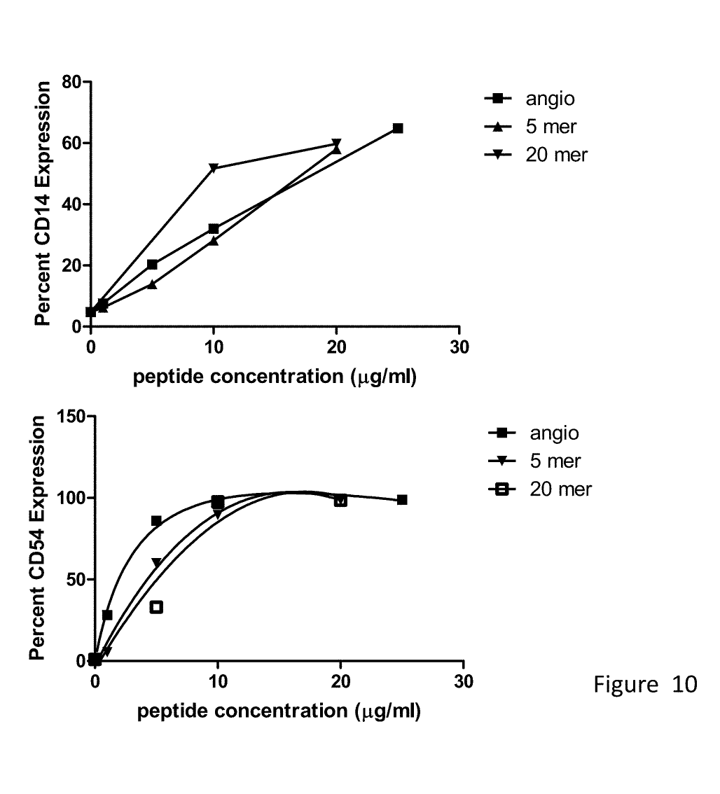

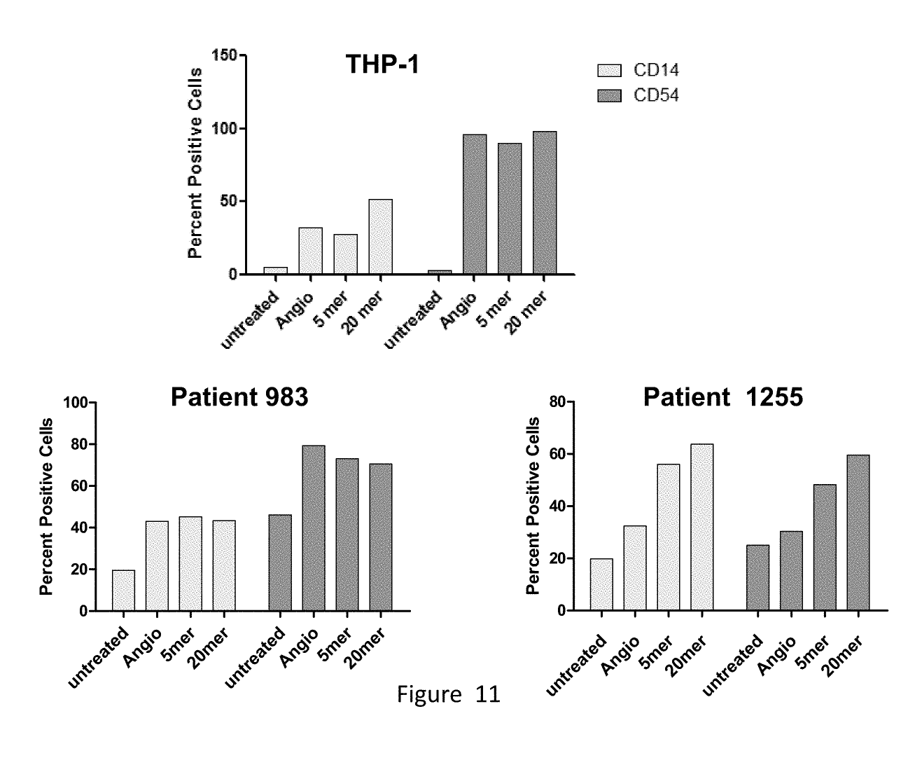

Experiments were also performed comparing the leukemia cell differentiating activity of the 5 mer, the 20 mer and angiocidin. These experiments show that the 5 mer, the 20 mer and angiocidin both upregulate CD54 in THP-1 leukemia cells (FIGS. 8-11) and that the 5 mer, 20 mer and angiocidin both upregulate a similar panel of cytokines from three different leukemia cell lines (FIGS. 8-11). These data showing that the 5 mer, 20 mer and angiocidin have nearly identical leukemia cell differentiating activities and therefore supports the use of the 5 mer and 20 mer in the clinical setting to mimic the anti-tumor activity of angiocidin.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An isolated peptide that interacts with death receptor-6 (DR6) and is capable of modulating DR6 activity, wherein the peptide comprises a sequence selected from the group consisting of SEQ ID NO: 1, a fragment of SEQ ID NO: 1, a variant of SEQ ID NO: 1, SEQ ID NO: 2, a fragment of SEQ ID NO: 2, a variant of SEQ ID NO: 2, and any combination thereof.

2. The peptide of claim 1, wherein the peptide variant of SEQ ID NO: 1 is at least about 75% homologous to SEQ ID NO: 1.

3. The peptide of claim 1, wherein the peptide variant of SEQ ID NO: 2 is at least about 75% homologous to SEQ ID NO: 2.

4. A method for modulating activity of death receptor-6 (DR6) on a cell, the method comprising contacting a cell with a peptide that interacts with DR6 and is capable of modulating DR6 activity, wherein the peptide comprises a sequence selected from the group consisting of SEQ ID NO: 1, a fragment of SEQ ID NO: 1, a variant of SEQ ID NO: 1, SEQ ID NO: 2, a fragment of SEQ ID NO: 2, a variant of SEQ ID NO: 2, and any combination thereof.

5. A method of treating or preventing a disease or condition in a subject in need thereof, wherein the disease or condition is associated with DR6 activity, the method comprising administering to the subject a therapeutically effective amount of a composition comprising a peptide that interacts with DR6 and is capable of modulating activating DR6 activity, wherein the peptide comprises a sequence selected from the group consisting of SEQ ID NO: 1, a fragment of SEQ ID NO: 1, a variant of SEQ ID NO: 1, SEQ ID NO: 2, a fragment of SEQ ID NO: 2, a variant of SEQ ID NO: 2, and any combination thereof, whereby administration of the composition to the subject treats or prevents the disease or condition in the subject.

6. The method of claim 5, wherein the disease or condition that is associated with DR6 activity is cancer.

7. The method of claim 6, wherein the cancer is selected from the group consisting of leukemia, glioma, breast cancer, melanoma, and any combination thereof.

8. A method of detecting DR6, the method comprising contacting DR6 with a peptide wherein the peptide comprises a sequence selected from the group consisting of SEQ ID NO: 1, a fragment of SEQ ID NO: 1, a variant of SEQ ID NO: 1, SEQ ID NO: 2, a fragment of SEQ ID NO: 2, a variant of SEQ ID NO: 2, and any combination thereof.

9. A kit for detecting DR6, the kit comprising an instruction manual and a peptide, wherein the peptide comprises a sequence selected from the group consisting of SEQ ID NO: 1, a fragment of SEQ ID NO: 1, a variant of SEQ ID NO: 1, SEQ ID NO: 2, a fragment of SEQ ID NO: 2, a variant of SEQ ID NO: 2, and any combination thereof.

10. A method of diagnosing a disease or condition associated with DR6 activity in a subject, the method comprising contacting a biological sample derived from a subject with a peptide, wherein the peptide comprises a sequence selected from the group consisting of SEQ ID NO: 1, a fragment of SEQ ID NO: 1, a variant of SEQ ID NO: 1, SEQ ID NO: 2, a fragment of SEQ ID NO: 2, a variant of SEQ ID NO: 2, and any combination thereof for a time sufficient to generate a DR6-peptide complex, wherein detection of the presence of the DR6-peptide complex diagnoses the subject with a disease or condition associated with DR6 activity.

11. The method of claim 10, wherein the disease or condition that is associated with DR6 activity is cancer.

12. The method of claim 11, wherein the cancer is selected from the group consisting of leukemia, glioma, breast cancer, melanoma, and any combination thereof.

13. An antibody that specifically binds to a peptide, wherein the peptide comprises a sequence selected from the group consisting of SEQ ID NO: 1, a fragment of SEQ ID NO: 1, a variant of SEQ ID NO: 1, SEQ ID NO: 2, a fragment of SEQ ID NO: 2, a variant of SEQ ID NO: 2, and any combination thereof.

14. An antibody that binds DR6 and prevents interaction with angiocidin or angiocidin-derived peptides.

15. The antibody of claim 14, wherein the antibody specifically binds to a peptide comprising a sequence selected from the group consisting of SEQ ID NO: 1, a fragment of SEQ ID NO: 1, a variant of SEQ ID NO: 1, SEQ ID NO: 2, a fragment of SEQ ID NO: 2, a variant of SEQ ID NO: 2, and any combination thereof.
